(19) United States
(12) Patent Application Publication
(10) Pub. No.: US 2015/0196634 A1
(43) Pub. Date: Jul. 16, 2015

(54) CULTURED MYELOID DENDRITIC CELLS ISOLATED FROM PEYERS PATCHES AND USES THEREOF

(71) Applicant: Institut National de la Sante et de la Recherche Medicale (INSERM), Paris (FR)

(72) Inventors: Jean-Pierre GORVEL, Marseille (FR); Hugues LELOUARD, La Destrousse (FR)

(21) Appl. No.: 14/668,112

(22) Filed: Mar. 25, 2015

Related U.S. Application Data
(63) Continuation of application No. 12/813,719, filed on Jun. 11, 2010.

(51) Int. Cl. A61K 39/35; A61K 39/39; A61K 45/06

(52) U.S. Cl. CPC A61K 39/35; A61K 45/06; A61K 39/39; A61K 2039/55588

(57) ABSTRACT

Cultured lysozyme-secreting myeloid dendritic cells isolated from the subepithelial dome of Peyer's patches in the small intestine are provided. The myeloid dendritic cells are used in screening methods to identify agents which interact with myeloid dendritic cells, for example, antigens, allergens, antimicrobial agents, or agents that modulate the activity of myeloid dendritic cells. The cells are also used to identify agents which bind to and/or are taken up by myeloid dendritic cells, and which can act as delivery vehicles for delivering substances of interest to myeloid dendritic cells in vivo.

### FIELD OF THE INVENTION

The present invention relates to a myeloid dendritic cell isolated from the subepithelial dome of Peyer's patches in small intestine of a subject. The myeloid dendritic cell secretes lysozyme and may be used in a variety of screening techniques.

### BACKGROUND OF THE INVENTION

Antimicrobial proteins are key effectors of innate immunity. Their abundant expression in specialized epithelial cells, phagocytes, and granulocytes is critical to protect mucosal surfaces from pathogens. Among these molecules, lysozyme is one of the best-characterized bactericidal factors. Lysozyme cleaves peptidoglycan, the bacterial cell wall polymer of both Gram-positive and Gram-negative bacteria, rendering bacteria susceptible to disruption by osmotic pressure. Nevertheless, lysozyme is active predominantly against Gram-positive bacteria whose peptidoglycan is easily accessible because of the absence of an outer membrane. The antimicrobial activity of lysozyme may also include activation of bacterial autolytic enzymes or membrane disruption. Lysozyme appears also to facilitate the inflammatory potential of peptidoglycan by increasing its solubility, clearance, and availability. For instance, lysozyme-deficient transgenic mice exhibit increased inflammation in Gram-positive infections and it is inferred that lysozyme production by intestinal Paneth cells also modulates peptidoglycan-induced inflammatory responses.

Because of the abundance of lysozyme in pulmonary secretions, most research has focused on its role in airway host defense; much less is known about the secretion and role of lysozyme in the gut. Nevertheless the small intestine has an extensive epithelial surface required for nutrient absorption and thus is another major site of entry for pathogenic bacteria. In the gut, lysozyme is mainly found in Paneth cells of the crypt epithelium with no or little expression in the villi.

Peyer's patches (PP) are aggregations of lymphoid tissue that are typically found in the lowest portion of the small intestine ileum in humans; as such, they differentiate the ileum from the duodenum and jejunum. PP comprise clustered domes formed by B-cell follicles that contain germinal centers (GC). Follicles are separated from each other by interfollicular regions (IFR), rich in T cells and dendritic cells (DC), and from the overlying epithelium by a subepithelial dome (SED) rich in DC. The follicle-associated epithelium (FAE) contains specialized epithelial cells, called M cells, that bind and rapidly transport several pathogens such as Salmonella Typhimurium from the lumen to an invagination of their basolateral membrane forming a pocket in which DC, macrophages, and T and B lymphocytes reside to trigger an appropriate mucosal immune response. However, expression of lysozyme in PP, which are major sites of antigen sampling and pathogen entry in the small intestine, has not been reported.

### SUMMARY OF THE INVENTION

The present invention relates to a myeloid dendritic cell isolated from the subepithelial dome of Peyer's patches in the small intestine. The isolated myeloid dendritic cell is characterized by its secretion of lysozyme. When cultured in vitro, the myeloid dendritic cell isolated from the subepithelial dome of Peyer's patches in the small intestine serves as a prototype of lysozyme secreting intestinal cells.

The cells of the invention may be used for a variety of purposes, for example, for a variety of screening methods and procedures, including high throughput screening (HTS). Screening procedures include those which are designed to identify: substances that have a particular desired effect on the properties or activities of myeloid dendritic cells (e.g. various candidate drugs); nucleic acids which, when introduced into and expressed by the myeloid dendritic cells, bring about a desired effect or cause a desired change in the properties or activities of the cell; or to identify substances or constructs that bind to myeloid dendritic cells (especially if the binding is selective or specific); and/or to identify substances or constructs which are readily taken up by myeloid dendritic cells, with or without exerting a particular effect on the cells. The identification of substances or constructs which are readily taken up by the myeloid dendritic cells of the invention (especially if the uptake is specific to myeloid dendritic cells) permits the production of delivery systems designed to deliver substances of interest selectively or specifically to myeloid dendritic cells in vivo. Such delivery systems generally comprise at least two components, one of which targets (e.g. binds to and is taken up by) myeloid dendritic cells, and the other of which is a substance of interest which is attached to or contained within the targeting component, and which is introduced or carried into the myeloid dendritic cells along with the targeting component when it is taken up by the cell. However, in some cases, the delivered agent or substance of interest may exert its effect without being internalized into the cell, e.g. the substance may act at the cell surface, or while tethered to the cell surface by the targeting moiety of the delivery system.

The invention provides a cultured myeloid dendritic cell isolated from the subepithelial dome of Peyer's patches in small intestine. The myeloid dendritic cell secretes lysozyme. In some embodiments, the cultured myeloid dendritic cell is isolated from a mammal, for example, a rodent (rat, mouse, etc.) or a human. In some embodiments, the cultured myeloid dendritic cell is CD11c⁺CD11b⁺CX3CR1⁺CD8a⁻: with respect to dendritic cell surface marker reactivity. In other embodiments, the myeloid dendritic cell is CD11c⁺CD11b^{Lo to Hi}CD8a⁻F4/80⁻CX3CR1⁺JAM-A⁺ with respect to dendritic cell surface marker reactivity. In yet other embodiments, the cultured myeloid dendritic cell is CD11c⁺BDCA1⁺ with respect to dendritic cell surface marker reactivity. In some embodiments of the invention, the cultured myeloid dendritic cell is genetically engineered to express at least one nucleic acid sequence encoding an expression product of interest.

The invention also provides a method for isolating a myeoloid dendritic cell. The method comprises the steps of 1) collecting a population of cells from the subepithelial dome of Peyer's patches in small intestine of a subject; 2) sorting dendritic cells in said population using antibodies to one or more surface markers of dendritic cells; testing sorted dendritic cells to identify cells which secrete lysozyme; and collecting dendritic cells which secrete lysozyme. In one embodiment, the one or more surface markers of dendritic cells is selected from the group consisting of CD11c, CD11b, CX3CR1, CD8a, F4/80, CX3CR1, JAM-A, and BDCA1. In another embodiment, the method further comprises a step of testing the sorted dendritic cells to identify cells which are capable of phagocytosis.

The invention further provides a method of screening one or more agents for an activity or property of interest based on the one or more agent's interaction with myeloid dendritic cells. The method comprises the steps of 1) exposing lysozyme-secreting myeloid dendritic cells isolated from the subepithelial dome of Peyer's patches in small intestine of a subject to said one or more agents; 2) determining whether said one or more agents interacts with said myeloid dendritic cells i) by causing said myeloid dendritic cells to increase or decrease lysozyme secretion; or ii) by binding to said myeloid dendritic cells; or iii) by being captured by said myeloid dendritic cells; and, based on said determining step; 3) concluding that one or more agents has or is likely to have said activity or property of interest.

In one embodiment, the activity or property of interest is for use against gut pathogens, for example, gut pathogens such as Mycoplasma, Mycobacteria, Legionella, Trypanosoma, Leislimanias, Listeria, Brucella and Salmonella. In another embodiment, the activity or property of interest is for use in treating bowel disease such as ulcerative colitis, Crohn's disease, inflammatory bowel diseases, pouchitis, collagenous colitis, irritable bowel syndrome, chronic constipation, chronic diarrhea, antibiotic-associated pseudomembranous colitis, diverticular disease of the colon, intestinally caused halitosis, polymorphous light eruption, non-ulcer dyspepsia, food intolerance, food malabsorptions, extra-intestinal Escherichia coli infections and mycoses of the orogastrointestinal tract. In yet another embodiment, the activity or property of interest is for use as an immunoadjuvant.

In some embodiments, when an agent is determined to be (iii) captured by the myeloid dendritic cells, the method further comprises the step of determining whether the agent is specifically captured by the myeloid dendritic cells. This determining step includes comparing (e.g. measuring or quantitating) capture of the agent by the myeloid dendritic cells to capture of the agent by at least one different type of cell.

In some embodiments of the invention, the activity or property of interest is for use as a stimulant of myeloid dendritic cells.

In some embodiments, the one or more agents are, for example compounds, cells, or microspheres. The compounds may be, for example, peptides, petptidomimetics, small organic molecules, antibodies, aptamers and nucleic acids.

In some embodiments of the invention, the activity or property of interest is for use as a probiotic, for example a probiotic such as Bacillus, Bifidobacterium, Lactobacillus, Streptococcus thermophilus and Escherichia coli.

The invention further encompasses a composition comprising 1) an agent that is able to be captured by a myeloid dendritic cell (the myeloid dendritic cell being isolated from the subepithelial dome of Peyer's patches in small intestine of a subject, and secreting lysozyme); and 2) a substance of interest bound to the agent. Substances of interest may be, for example, antigens, allergens, tolerogens, adjuvants, drugs, chemicals, DNA, RNA, expression vector systems, engineered viruses, toxins, and enzymes; anti-tumor agents or an anti-infection agents (e.g. anti-microbial or anti-parasitic agents); or food allergens.

In yet another embodiment, the invention provides a method of delivering a substance of interest to a subject. The method comprises the step of administering to the subject an agent which binds to or is taken up by lysozyme-secreting myeloid dendritic cells isolated from the subepithelial dome of Peyer's patches, the substance of interest being associated with said agent. Exemplary substances of interest include antigens, allergens, tolerogens, adjuvants, drugs, chemicals, DNA, RNA, expression vector systems, engineered viruses, toxins, and enzymes.

### DETAILED DESCRIPTION OF THE INVENTION

In the gastrointestinal tract, lysozyme is thought to be mainly expressed by Paneth cells of the crypt epithelium. The inventors thus investigated its expression in the Peyer's patch, a major intestinal site of antigen sampling and pathogen entry. Immunostaining was performed on normal and Salmonella Typhimurium-infected intestinal samples and the samples were analyzed by confocal microscopy and flow cytometry. In Peyer's patch of mouse, rat, and human, lysozyme was strongly expressed in the germinal center of follicles by tingible body macrophages and in the subepithelial dome by a subset of myeloid dendritic cells (DC). Among DC subsets from mouse Peyer's patches, these lysozyme-expressing DC displayed the highest surface expression of class II major histocompatibility complex and co-stimulatory molecules, and they were the most efficient at capturing microspheres in vitro. Moreover, they were the main DC subset involved in bacterial pathogen uptake and in dead cell clearance, including M cells. The subepithelial dome of Peyer's patches thus contains a unique population of intestinal DC that secretes high levels of lysozyme and internalizes bacteria and dead cells.

The present invention thus relates to a myeloid dendritic cell isolated from the subepithelial dome of Peyer's patches in small intestine of a subject. The cells are characterized in that these myeloid dendritic cells secrete lysozyme.

As used herein, the term “myeloid dendritic cell” has its general meaning in the art. The myeloid dendritic cell is an antigen presenting cell existing in vivo, in vitro, ex vivo, or in a host or subject, or which can be derived from a hematopoietic stem cell, a hematopoietic progenitor or a monocyte. The myeloid dendritic cell has a characteristic morphology with thin sheets (lamellipodia) extending in multiple directions away from the dendritic cell body. The main surface markers of human myeloid dendritic cells include CD11b, CD11c, CD33 CD115DCs and BDCA1. The myeloid dendritic cells express constitutively both MHC class I and class II molecules, which present peptide antigens to CD8+ and CD4+ T cells respectively. The myeloid dendritic cell membrane is also rich in molecules that allow adhesion of T cells (e.g. intercellular adhesion molecule 1 or CD54) or that co-stimulate T-cell activation such as B7-1 and B7-2 (also known as CD80 and CD86 respectively).

By “isolated” it is meant, when referring to a cell or a population of cells that said cell or said population of cells is present in the substantial absence of other cells or population of cells.

As used herein, the term “lysozyme” has its general meaning in the art. Lysozyme, also known as murarnidase or N-acetylmuramide glycanhydrolase, is a family of enzymes (EC 3.2.1.17) which damage bacterial cell walls by catalyzing hydrolysis of 1,4-beta-linkages between N-acetylmuramic acid and N-acetyl-D-glucosamine residues in a peptidoglycan and between N-acetyl-D-glucosamine residues in chitodextrins.

As used herein, the term “subject” denotes a vertebrate, preferably a mammal, such as a rodent, e.g. a mouse; a feline, a canine, and a primate. In exemplary embodiments, a subject according to the invention is a human or a rodent.

Typically, when the subject is a mouse, the myeloid dendritic cell according to the invention is CD11c⁺CD11b⁺CX₃CR₁⁺CD8a⁻, when characterized by antibody binding to surface markers of dendritic cells. More particularly, mouse myeloid dendritic cells according to the invention are CD11c⁺CD11b^{Lo to Hi}CD8a⁻F4/80⁻CX3CR1⁺JAM-A⁺.

Typically, when the subject is a human, the myeloid dendritic cell according to the invention is CD11c⁺BDCA1⁺.

The myeloid dendritic cells according to the invention may be isolated according to any technique known in the art.

For example, one standard method for isolating myeloid dendritic cells according to the invention involves collecting a population of cells from the subepithelial dome of Peyer's patches in the small intestine of a subject and then using different antibodies specific for surface markers characteristic of dendritic cells (e.g. CD11c+), and finally testing whether said cells secrete lysoszyme. The method may also further comprise a step of testing whether the cells are capable of phagocytosis.

Typically, fluorescence activated cell sorting (FACS) may be used to separate the desired cells expressing selected surface marker from the population of isolated cells. In another embodiment, magnetic beads may be used to isolate dendritic cells from a cell population (MACS). For instance, magnetic beads labelled with monoclonal cell type specific antibodies may be used for the positive selection of the myeloid dendritic cells of the invention. Other methods can include the isolation of myeloid dendritic cells by depletion of non-dendritic cells (negative selection). Kits for isolation of myeloid dendritic cells are commercially available from Miltenyi Biotec (Auburn, Calif., USA), Stem Cells Technologies (Vancouver, Canada) or Dynal Bioech (Oslo, Norway).

For example, for a mouse subject, myeloid dendritic cells of the invention may be isolated from Peyer's patches, as previously described for lymph nodes DC (Henri S, Vremec D, Kamath A, et al. The myeloid dendritic cell populations of mouse lymph nodes. J Immunol 2001; 167:741-8.). Light-density cells may be then purified by centrifugation on a Nycoprep solution (density of 1.068). Finally, CD11c+ cells may be sorted using anti-CD11c microbeads and an AutoMACS magnetic cell separator according to manufacturer's instructions (Miltenyi Biotec GmBH).

Testing whether the isolated myeloid dendritic cells secrete lysozyme may be readily performed by one of skill in the art. Typically, lysozyme secretion may be detected by ELISA or immunostaining using antibodies directed to lysozyme.

In one embodiment, the present invention relates to a method for isolating a myeloid dendritic cell of the invention according to any of the above described methods. More particularly, said method comprises the steps of:

i) providing a sample of the subepithelial dome of Peyer's patches in small intestine of a subject
ii) purifying light density cells from the sample by centrifugation
iii) sorting CD11c+ cells from the light density cells obtained at step ii)
iv) selecting the CD11c+ cells obtained at step iii) that secrete lysozyme.

In particular the method as above described may further comprise (e.g. before step iv), a step of selecting the CD11c+ cells obtained in step iii) that phagocytose microspheres.

In one embodiment, the myeloid dendritic cells according to the invention may be genetically engineered so that said cells express at least one sequence encoding an expression product. As used herein, a “sequence encoding an expression product” such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon. Typically, said nucleic acid is a DNA or RNA molecule and is delivered to the myeloid dendritic cell of the invention using a suitable vector, such as a plasmid, cosmid, episome, artificial chromosome, phage, viral vector, etc.

As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

Any expression vector for animal cells can be used, as long as a gene encoding a product of interest can be inserted and expressed. Examples of suitable vectors include but are not limited to pAGE107, pAGE103, pHSG274, pKCR, pSG1 beta d2-4 and the like. Other examples of plasmids include but are not limited to replicating plasmids comprising an origin of replication, or integrative plasmids, such as, for instance, pUC, pcDNA, pBR, and the like. Other examples of viral vectors include but are not limited to adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include but are not limited to PA317 cells, PsiCRIP cells, GPenv+ cells, 293 cells, etc. Detailed protocols for producing such replication-defective recombinant viruses may be found for instance in WO 95/14785, WO 96/22378, and WO 94/19478; and U.S. Pat. No. 5,882,877, U.S. Pat. No. 6,013,516, and U.S. Pat. No. 4,861,719, U.S. Pat. No. 5,278,056, the complete contents of each of which is hereby incorporated by reference.

Examples of promoters and enhancers used in the expression vector for animal cells include but are not limited to the early promoter and enhancer of SV40, LTR promoter and enhancer of Moloney mouse leukemia virus, promoter and enhancer of immunoglobulin H chain and the like.

The invention also encompasses the use of non viral gene delivery systems in order to introduce substances of interest into the cells of the invention. Such non viral systems generally comprise at least one nucleic acid molecule and a non-viral gene delivery vehicle. Examples of non viral gene delivery vehicles include but are not limited to liposomes and polymers such as polyethylenimines, cyclodextrins, histidine/lysine (HK) polymers, etc.

Typically, the myeloid dendritic cells of the invention can be engineered to inhibit expression of genes by siRNA or antisense encoding genes that are stably or transiently introduced into the cells. Targets for inhibition include but are not limited to inflammatory cytokines, proteases, transcription factors and enzymes affecting inflammatory pathways, etc.

In addition, myeloid dendritic cells of the invention can be genetically engineered to express one or more growth factors that promote differentiation and/or proliferation.

Myeloid dendritic cells of the invention may be further engineered so that said cells carry one or more molecules of interest. Suitable molecules (or even genes) of interest include but are not limited to protease inhibitors or agents promoting knockdown of proteases, transcription factors or dominant versions thereof to globally inhibit expression of inflammatory mediators, cytokines, chemokines, proteases or to globally induce anti-inflammatory mediators, cytokines, chemokines and protease inhibitors, etc.

In one embodiment, myeloid dendritic cells of the invention can be genetically engineered with one or more sequences encoding an expression product that is labelled with a detectable molecule. Typically, this system (i.e. a genetically engineered myeloid dendritic cell as described herein) is particularly suitable for analysing whether such a cell expresses a particular expression product under a particular condition (e.g. when contacted with a candidate as defined below).

According to the invention, said detectable molecule may consist of any compound or substance that is detectable by spectroscopic, photochemical, biochemical, immunochemical, chemical, or other suitable means. For example, useful detectable molecules include radioactive substance (including those comprising ³²P, ²⁵S, ³H, or ¹²⁵I, etc.), fluorescent dyes (including, for example, 5-bromodesosyrudin, fluorescein, acetylaminofluorene or digoxigenin), fluorescent proteins (including, for example, GFPs and YFPs), or detectable proteins or peptides (including biotin, polyhistidine tails or other antigen tags like the HA antigen, the FLAG antigen, the c-myc antigen and the DNP antigen, etc).

For any in vitro purpose, (e.g. screening methods as described below), the myeloid dendritic cells of the invention may be cultured according to any method known in the art. Typically, the myeloid dendritic cells of the invention may be disseminated (i.e. plated) in a suitable culture container. There is no particular limitation for the container used; a plate, laboratory dish, flask, bag or the like normally employed in cultivation in the art may be used. The concentration of the individual cell subsets disseminated may be freely set in accordance with the situation. “Cultured” myeloid dendritic cells of the invention are thus myeloid dendritic cells which have been removed from a source such as a mammalian subject, and maintained and usually propagated in vitro. The invention encompasses both the originally isolated cells (typically after selection and or characterization, as described herein) and also progeny of those cells which result from cell division of the original cells, while the cells are maintained and passaged in vitro. For use in the methods described herein, the cells may be grown on a substrate, e.g. a glass slide, on beads, in multiwell plates, in tubes, or in or on other containers or substrates suitable for carrying out the methods, e.g. for screening of various agents or substances of interest.

AIM-V® medium commercially available by Invitrogen™ may be used to culture the myeloid dendritic cells of the invention. In addition to AIM-V medium, any commercially available culture medium used in cell cultivation, such as RPMI-1640 medium, DMEM, TIL, KBM, and IMEM, etc., may be used. Moreover, 5-20% bovine serum, FBS, human plasma, cytokine or the like may be added as needed. The cultivation is carried out under the conditions of 34-38° C., more preferably 37° C., and 2-10% CO₂, more preferably 5% CO₂; the cultivation duration is preferably 5-8 days, more preferably 7 days.

Myeloid dendritic cells according to the invention may be suitable for use in a variety of screening methods. This aspect of the invention takes advantage of the ability of isolated or cultured cells of the invention to mimic characteristics, reactions and activities of in vivo cells, i.e. cells which are located within a living subject. Use of the cells of the invention is thus advantageous, for example, as a first step in screening drug candidates prior to in vivo testing.

For example, myeloid dendritic cells according to the invention are suitable in methods for screening drugs against pathogens present in the gut of a subject. For example, several pathogens are able to subvert the phagocytose process by a range of stratagems utilizing a vacuolar pathway for invasion and survival, even in macrophages. Other examples are known by which organisms evade ingestion (mycoplasma), destroy opsonins enzymatically, inhibit fusion and acidification (mycobacteria) and recruit novel membranes (Legionella) as well as other organelles. Trypanosoma cruzi and Candida albicans rapidly recruit lysosomes, perhaps to promote their own differentiation. Leishmania multiplies freely within phagolysosomes whereas Listeria monocytogenes disrupts lysosomal membranes and escapes into the cytoplasm, where it initiates actin polymerization for intracellular movement and intercellular spread. Bacteria of the genus Brucella are intracellular pathogens capable of survival and replication within macrophages of mammalian hosts. This pathogen uses multiple strategies to circumvent macrophage defence mechanisms and generate an organelle permissive for replication. Finally, the facultative intracellular pathogen Salmonella enterica triggers programmed cell death in macrophages.

Therefore the myeloid dendritic cells according to the invention may be useful for screening drugs against pathogens such as Mycoplasma, Mycobacteria, Legionella, Trypanosoma, Leishmanias, Listeria, Brucella or Salmonella, etc.

In another embodiment, the myeloid dendritic cells of the invention may be particularly suitable for screening drugs for the treatment diseases which include but are not limited to bowel diseases such as ulcerative colitis, Crohn's disease, inflammatory bowel diseases, pouchitis, collagenous colitis, irritable bowel syndrome, chronic constipation, chronic diarrhea, antibiotic-associated pseudomembranous colitis, diverticular disease of the colon, intestinally caused halitosis, polymorphous light eruption, non-ulcer dyspepsia, food intolerance, food malabsorptions, extra-intestinal E. coli infections and mycoses of the orogastrointestinal tract, etc. Indeed, without wishing to be bound by any, particular theory, it is believed that the myeloid dendritic cells may represent a first line of defence against microbial intruders present in the intestinal tract, but may also be involved in the physiology of intestinal diseases such as inflammatory bowel diseases, e.g. ulcerative colitis or Crohn's disease. Accordingly, the cells of the invention may be used for screening drugs useful for the treatment of the above mentioned diseases.

In another embodiment, the myeloid dendritic cells of the invention are also particularly suitable for in methods for screening immunoadjuvants. As used herein, the tem “immunoadjuvant” refers to a compound that can induce and/or enhance the immune response against an antigen when administered to a subject such as an animal. It is also intended to mean a substance that acts generally to accelerate, prolong, or enhance the quality of specific immune responses to a specific antigen. In an exemplary embodiment, said screening methods may evaluate the ability of a candidate drugs (e.g. compounds or agents) to modulate the secretion of lysozyme by said myeloid dendritic cell. Accordingly, in a particular aspect, said screening method may comprise the steps of:

i) contacting a myeloid dendritic cell of the invention with a candidate compound or agent;

ii) determining whether said candidate compound or agent increases or decreases the secretion of lysozyme by said myeloid dendritic cell; and
iii) selecting the candidate compound or agent that increases or decreases the secretion of lysozyme by said myeloid dendritic cell.

Candidate compounds or agents that have the ability to modulate the secretion of lysozyme may represent drugs particularly suitable against pathogens such as Mycoplasma, Mycobacteria, Legionella, Trypanosoma, Leishmanias, Listeria, Brucella, Salmonella, etc.

In another exemplary embodiment, the screening methods of the invention may also be used to evaluate the ability of a candidate compound or agent to target the myeloid dendritic cell of the invention.

Typically, said screening methods may, for example, evaluate the ability of the candidate compound or agent to bind the surface of the myeloid dendritic cells. Said binding may be measured by any known method in the art such as, for example, surface plasmon resonance (SPR). Accordingly, in a particular embodiment, the screening methods of the invention may comprise the steps of:

i) contacting a myeloid dendritic cell of the invention with a candidate compound or agent;

ii) determining whether said candidate compound or agent binds to said myeloid dendritic cell; and

iii) selecting the candidate compound or agent that binds to said myeloid dendritic cell. In particular the ability of the candidate to specifically bind the myeloid dendritic cells of the invention may also be evaluated by comparing the binding of the candidates to the myeloid dendritic cells of the invention and the binding of the candidates to another different cell type. Said other cell type may be, for example, another subset of myeloid dendritic cells, and more preferably another subset of myeloid dendritic cells that can be isolated from the subepithelial dome of Peyer's patches in small intestine of the subject. Candidates that specifically bind to the myeloid dendritic cells of the invention are preferably selected.

In another embodiment, the screening methods may further evaluate the ability of the candidate to be captured (i.e. taken up, phagocytised, etc.) by the myeloid dendritic cells of the invention. Said evaluation may be performed by the labelling the compound with a detectable molecule as above described, and then detecting said labelled candidate within the myeloid dendritic cell of the invention, and more preferably within a particular compartment of the myeloid dendritic cell of the invention (DAPI, cytoplasm, nucleus, etc.). Accordingly, in an exemplary embodiment, the screening methods of the invention may comprise the steps of:

i) contacting a myeloid dendritic cell of the invention with a candidate compound or agent;

ii) determining whether said candidate compound or agent is captured by said myeloid dendritic cell; and

iii) selecting the candidate compound or agent that captured by myeloid dendritic cell.

In particular, the ability of the candidate to be specifically captured into the myeloid dendritic cells of the invention may also be evaluated by comparing the capture of the candidates into the myeloid dendritic cells of the invention with the capture of the candidates into another cell type. Said another cell type may be for example another subset of myeloid dendritic cell, and more preferably another subset of myeloid dendritic cell that can be isolated from the subepithelial dome of Peyer's patches in small intestine of the subject. Candidates that are specifically captured by the myeloid dendritic cells of the invention are preferably selected. The capture of the candidate may occur through any cell mechanism, including but not limited to receptor internalization, endocytosis or phagocytosis, etc.

The capability of the candidate to be captured by the myeloid dendritic cells of the invention may be examined by standard methods, e.g. by labelling the candidate and tracing its fate (uptake into the myeloid dendritic cell). The candidate to be tested can be labelled (e.g. using a radioactive compound or a fluorochrome, or a toxin, or a drug influencing the metabolism of the myeloid dendritic cells of the invention) and reacted for an optimal time with the myeloid dendritic cell at a temperature at which capture can occur. After a sufficient incubation period, the rate of capture can be determined either by measuring the amount of the captured candidate by optical methods (in the case of a fluorophore-marker) or by measuring the incorporated radioactivity (in case of a radioactive marker such as [¹²⁵I]), or by assessing cell death (in case of a toxin), or by any other detection method suitable for the marker used.

“Selective” binding or uptake is characterized by the agent preferentially binding to the cells of the invention or being preferentially taken up by the cells of the invention by a factor of at least about 2-10 fold, or greater (e.g. even 100 or 1000 fold greater), than to a cell which is used for comparison. Specific binding or uptake refers to an essentially complete lack of binding to or uptake into the cell which is used for comparison, whereas at least some binding affinity for or uptake into the cells of the invention is detectable, using methods of analysis such as those described herein.

In another embodiment, the screening methods of the invention may evaluate the ability of a candidate compound or agent to stimulate the myeloid dendritic cell of the invention. Stimulation of the myeloid dendritic cells of the invention may result in a production of specific cytokines and/or specific surface marker that may participate in the ability of the myeloid dendritic cell to stimulate other cell types such as T or B cells. In a particular embodiment, the candidate may be assayed for its ability to induce production of one or more cytokines such as IL-12p, TNF-alpha, etc. In another exemplary embodiment, the candidate may be assayed for its ability to induce expression of specific surface markers such as one or more of CD80, CD40 and CD86, etc. In addition, the screening method may further comprise a step of evaluating the ability of the myeloid dendritic cells of the invention stimulated by the candidate compound to stimulate specific 1′ or B cell responses. The T cell response may be of any nature and includes typically, TCD4 response, TCD8 response or even Treg response (i.e. tolerogen response), etc. The B cell response may be of any type, including production of one or more of IgG, IgM and IgA, etc. Methods for the detection of stimulated myeloid dendritic cells, T cells and B cells, etc. are known to the skilled person. The person skilled in the art can indeed construe that any method suitable for assessing the stimulation of myeloid one or more of dendritic cells, T cells, and/or B cells, etc., in response to a candidate. Such methods include Enzyme-linked immunospot (ELISpot). Non-adherent cells from pre-culture wells are transferred to a plate which has been coated with the desired anti-cytokine capture antibodies (Abs; e.g., anti-cytokine antibodies). Analysis is carried out with biotinylated secondary Abs and standard colorimetric or fluorimetric detection methods such as streptavidin-alkaline phosphatase and NBT-BCIP and the spots counted. ELISpot readouts are then expressed as spot-forming cells (SFC)/10⁶ PBMCs. Cytokines released in the culture supernatant may also be measured by different techniques, such as enzyme-linked immunosorbent assays (ELISA), BD cytometric bead array, Biorad Bio-Plex assay and others.

According to the invention, the candidate that may be used in the screening methods may be of any nature for example: compounds (e.g. chemically synthesized small molecules, isolated or synthesized natural products, etc.), cells, microspheres, etc. Exemplary compounds include but are not limited to: peptides, petptidomimetics, small organic molecules, antibodies, aptamers or nucleic acids, etc. Other non-limiting examples include compounds selected from a library of compounds previously synthesised, or a library of compounds for which the structure is determined in a database, or from a library of compounds that have been synthesised de novo.

In one embodiment, the compound is a small organic molecule. As used herein, the term “small organic molecule” refers to a molecule of size comparable to those organic molecules generally used in pharmaceuticals. The term excludes biological macromolecules (e.g.; proteins, nucleic acids, etc.); preferred small organic molecules range in size up to about 2000 da, and most preferably up to about 1000 Da.

In another embodiment, the compounds are antibodies or functional derivatives thereof. The antibodies may be preferably monoclonal antibodies or an antigen-binding fragment thereof, e.g. of the F (ab′)2, or single chain Fv type, or any type of recombinant antibody derived therefrom. The antibodies of the invention can, for instance, be produced by any hybridoma produced according to classical methods from splenic cells of an animal, particularly of a mouse or rat immunized against an antigen, and of cells of a myeloma cell line, and selected by the ability of the hybridoma to produce monoclonal antibodies recognizing the antigen which was initially used for the immunization of the animals. The antibodies according to this embodiment of the invention may be humanized versions of the mouse antibodies made by means of recombinant DNA technology, departing from the mouse and/or human genomic DNA sequences coding for H and L chains or from cDNA clones coding for H and L chains. Alternatively the antibodies according to this embodiment of the invention may be human antibodies. Such human antibodies are prepared, for instance, by means of human peripheral blood lymphocytes (PBL) repopulation of severe combined immune deficiency (SCID) mice as described in PCT/EP 99/03605 or by using transgenic non-human animals capable of producing human antibodies as described in U.S. Pat. No. 5,545,806, the complete contents of which is hereby incorporated by reference. Also, fragments derived from these antibodies such as Fab, F (ab)′2 or the “single chain variable fragment” type, providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies or fragments thereof, can be modified for various uses. An appropriate label of the enzymatic, fluorescent, or radioactive type can label the antibodies involved in the invention.

In another embodiment, the compounds that are screened may be aptamers. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L. (Science, August 3; 249(4968):505-10, 1990). The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli thioredoxin A that are selected from combinatorial libraries by two hybrid methods. In one embodiment, the antibodies or aptamers according to the invention may be directed to a surface protein of the myeloid dendritic cell of the invention.

Also within the scope of the invention is the screening of oligoribonucleotide sequences that include anti-sense RNA and DNA molecules and ribozymes that function to inhibit the translation of a particular mRNA. Anti-sense RNA and DNA molecules act to directly block the translation of mRNA by binding to targeted mRNA and preventing protein translation. In regard to antisense DNA, oligodeoxyribonucleotides derived from the translation initiation site. Ribozymes are enzymatic RNA molecules capable of catalysing the specific cleavage of RNA. The mechanism of ribozyme action involves sequence specific hybridisation of the ribozyme molecule to complementary target RNA, followed by an endonucleolytic

In an exemplary embodiment, the candidate that is screened may be a cell (i.e. a eukaryote or prokaryote cell). In one embodiment the cell is a bacterium and more particularly a probiotic.

As used herein, the term “probiotic” refers to a live microorganism which when administered in adequate amounts confers a health benefit on the host. Probiotics are thought to provide to the host beneficial effects such as reducing inflammation, preventing harmful bacterial growth under stress, preventing inflammatory bowel diseases such as Crohn's disease or ulcerative colitis, or improving immune function and preventing infections. Regarding theses effects, screening methods of the invention may be particularly suitable to test whether said probiotic indeed interacts with (e.g. stimulation, capture, etc.) the myeloid dendritic cells of the invention. Alternatively, the screening methods of the invention may also particularly suitable for selecting appropriate probiotics that are able to interact (e.g. stimulation, capture . . . ) with the myeloid dendritic cells of the invention. According to the invention, the probiotic strain may be typically selected from the group consisting of Bacillus (e.g. Bacillus coagulans), Bifidobacterium, Lactobacillus (i.e. Lactobacillus acidophilus or Lactobacillus casei), Streptococcus thermophilus or Escherichia coli.

In one embodiment, the candidate that is screened may be a microsphere. As used herein the term “microsphere” refers to a spherical shell that is usually made of a biodegradable or resorbable plastic polymer, that has a very small diameter usually in the micrometer or nanometer range, and that is often filled with a substance (as a drug or antibody) for release as the shell is degraded. Screened using the myeloid dendritic cells of the invention permits identification of microspheres which are especially suitable for use in delivering substances of interest to myeloid dendritic cells, e.g. to in vivo cells located in a subject that is treated by the methods described herein.

In yet another embodiment, the screening methods of the invention may be useful for manufacturing a delivery system suitable for delivering a substance into a myeloid dendritic cell, e.g. in the intestines. The delivery system according to the invention may comprise i) a candidate that can be captured by the dentritic cell of the invention as described above; and ii) a substance to be delivered, wherein the substance is bound to the candidate. In an exemplary embodiment, the substance to be delivered may be, for example, an antigen, allergen, tolerogen, adjuvant, drug, chemical, DNA, RNA, expression vector system, engineered virus, toxin, enzyme, etc.

In one embodiment, the substance to be delivered is an antigen. The term “antigen” (“Ag”) as used herein refers to protein, peptide, tissue or cell preparations capable of eliciting a T cell response. In one embodiment, said Ag is a protein which can be obtained by recombinant DNA technology or by purification from different tissue or cell sources. Such proteins are not limited to natural ones, but also include modified proteins or chimeric constructs, obtained for example by changing selected amino acid sequences or by fusing portions of different proteins. In another embodiment of the invention, said Ag is a synthetic peptide, obtained, for example, by Fmoc biochemical procedures, large-scale multipin peptide synthesis, recombinant DNA technology or other suitable procedures.

In one embodiment, said Ag is a protein which can be obtained by recombinant DNA technology or by purification from various tissue or cell sources. Typically, said protein has a length greater than 10 amino acids, preferably greater than 15 amino acids, even more preferably greater than 20 amino acids, with no theoretical upper limit. Such proteins are not limited to natural ones, but also include modified proteins or chimeric constructs, obtained, for example, by changing selected amino acid sequences or by fusing portions of different proteins, polypeptides or peptides.

In another embodiment of the invention, said Ag is a synthetic peptide. Typically, said synthetic peptide is 3-40 amino acids in length, preferably 5-30 amino acids in length, even more preferably 8-20 amino acids in length. Synthetic peptides can be obtained, for example, by Fmoc biochemical procedures, large-scale multipin peptide synthesis, recombinant DNA technology or other suitable procedures. Such peptides are not limited to natural ones, but also include modified peptides or chimeric peptides, obtained, for example, by changing selected amino acid sequences or by fusing portions of different proteins, polypeptides or peptides.

In another embodiment of the invention, the Ag is a crude or partially purified tissue or cell preparation obtained by any of various biochemical procedures (e.g., fixation, lysis, subcellular fractionation, density gradient separation) known to the expert in the art.

In yet another embodiment, the substance to be delivered may be an allergen. An allergen is a substance capable of producing hypersensitivity or an allergic reaction. Usually, it comprises a nonpathogen-derived antigen capable of stimulating a hypersensitivity reaction in individuals. Typically, the allergen according to the invention is a food allergen that may be, for example, extracted from e.g. egg, peanut, milk, shellfishes, fishes, wheat, tree nut, soy or seeds, etc.

In another embodiment, the substance to be delivered may be a tolerogen. A tolerogen is an immunogen that stimulates an immune response, but does not invoke an inflammatory immune defence reaction. It may be used to induce tolerance in the immune system against its components.

In another embodiment, the substance to be delivered may be a toxic agent. For example, the toxic agent may be a cytotoxin, an apoptosis inducing agent, a ribosome-inactivating agent, a DNA- or RNA-cleaving agent, or an inhibitor of protein synthesis, etc. A cytotoxin is a substance having a direct toxic or destructive effect on certain cells of the body (usually those of a particular organ). Many chemotherapeutic drugs work by impairing mitosis, effectively targeting fast-dividing cells. Examples of common chemotherapeutics are alkylating agents (such as cisplatin, carboplatin and oxaliplatin), antimetabolites (e.g. those masquerading as purine ((azathioprine, mercaptopurine)) or pyrimidine), anthracyclines, plant alkaloids (such as vinca-alkaloids and taxanes) and topoisomerase inhibitors (such as irinotecan, topotecan, amsacrine, etoposide, etoposide phosphate, and teniposide) affecting cell division or DNA synthesis. Further chemotherapeutics acting in a different manner include monoclonal antibodies (targeting tumor-specific antigens (such as trastuzumab (Herceptin), cetuximab, and rituximab) or blocking formation of new tumor vessels (such as bevacizumab (Avastin)) and the new tyrosine kinase inhibitors e.g. imatinib mesylate (Gleevec® or Glivec®), which directly targets a molecular abnormality in certain types of cancer (chronic myelogenous leukemia, gastrointestinal stromal tumors). Functionally, the toxin may also be an apoptosis-inducing agent (an agent inducing programmed cell death of a cell such as gemcitabine, TNF-related apoptosis-inducing ligand (TRAIL) or an adamantyl group-containing retinoid-related compound), a ribosome-inactivating agent (a large group of toxic proteins widely distributed among the plant kingdom and inactivating ribosomes, e.g. by enzymatically attacking the 60S subunit of eukaryotic ribosomes and irreversibly modifying its large ribosomal RNA (rRNA) such as ricin, aviscumine, or a Shiga-like ribosome inactivating protein), a DNA- or RNAcleaving agent (i.e. a DNA/RNA interactive compound that binds to and cleave DNA/RNA such as a 1,2,4-benzotriazine 1,4-dioxide, resveratrol, cisplatin or hammerhead ribozyme) or an inhibitor of protein synthesis (a compound which inhibits the synthesis of proteins by e.g. interruption of peptide-chain elongation, blocking site of ribosomes, misreading of the genetic code or prevention of the attachment of oligosaccharide side chains to glycoproteins such as antibiotics (e.g. anisomycin, chloramphenicol, streptomycin, tetracycline, neomycin or erythromycin) fusidic acid, diptheria toxin, ricin or cycloheximide, etc.

For example, the substance ii) of the delivery system (including e.g. immunogen, allergen, tolerogen, adjuvant, drug, chemical, DNA, RNA, expression vector system, engineered virus, toxin, enzyme, etc.) can be associated with a candidate, e.g. by being non-covalently attached to the candidate that can be captured by the myeloid dendritic cell of the invention (i.e. the targeting agent), or by ionic strength forces, adhesion, cohesion, and/or by other molecular forces. Alternatively, and preferably, the substance can be directly linked to the candidate by chemical coupling, or utilizing a linker such as a peptide linker, or as a fusion protein in case of proteinacious components. Alternatively, the substance to be delivered (for example the immunogen, allergen, tolerogen, adjuvant, drug, chemical, DNA, RNA, expression vector system, engineered virus, toxin, enzyme, etc.) could be packaged/encapsulated into a “vehicle” to preserve the integrity and effectiveness of the substance to be targeted to the myeloid dendritic cell of the invention. Such a vehicle could be a live or dead cell, virus, virus-like particle, nanoparticle. lipid-based system (e.g. liposome), exosome, apoptotic body, colloidal dispersion system, polymer, carbohydrate, microsphere, or any other suitable vehicle. This vehicle would be targeted to the myeloid dendritic cell of the invention by the presence of a candidate that can be captured by the myeloid dendritic cells of the invention. More particularly, the candidate is present at the (outer) surface of the vehicle, in order to allow a specific binding of the vehicle to the myeloid dendritic cells of the invention, followed by capture, if required. In some cases, the delivered agent may exert its effect without being internalized into the cell. A particularly preferred vehicle is a structural protein of a virus or a multimeric structure thereof, such as a capsomere, a virus like particle or a virus. The multimeric structure may be an aggregate of at least about 5, preferably at least about 10, more preferably at least about 30, most preferably at least about 60 structural proteins and may contain the substance to be delivered inside the multimeric structure. It is known that a structural protein of viruses such as parvoviruses (e.g. adeno-associated virus 2) may be modified to present on their surface a particular protein.

Accordingly, the delivery system according to the invention may be useful for any therapeutic purposes, including but not limited to as a vaccine, or for immunogenic or tolerogenic purposes.

A vaccine targeted to the myeloid dendritic cells of the invention and could be used to elicit an immune response in or to immunize healthy individuals to protect them from infection (“protective vaccine”). Such a vaccine could be used for therapeutic purposes. As targeted immunogens may include attenuated, or dead pathogens, i.e. viruses, bacteria, parasites, fungi, mycoplasma, inactivated toxins, or immunogenic components thereof. The immunogen can also be applied as a protein or peptide moiety inducing immunity to the pathogen. For an immunogenic vaccine, a simultaneous application of a “danger signal”-type adjuvant is in general necessary, unless the pathogen or its component already provides the necessary “danger signal”. Such a danger signal” could be provided by a variety of components, examples are LPS, unmethylated CpG, High Mobility Group Protein Bl, heat-shock proteins, and other, see above). This approach can be applied to a variety of pathogens. Examples of pathogens that can be targeted using the cells of the invention include but are not limited to various Mycoplasma, Mycobacteria, Legionella, Trypanosoma, Leishmanias, Listeria, Brucella or Salmonella species, etc.

The vaccine could also be used to protect healthy individuals from developing tumors with known antigenic components (“tumor protective vaccine”). In such a case the patient would be treated with known tumor antigens or his own (excised) tumor material targeted in such a fashion to the myeloid dendritic cell of the invention, as to elicit a powerful cytotoxic ThI immune response against tumor-specific antigens.

Some vaccines may also be used for desensitization of allergic individuals. Allergic individuals are prone to develop a Th2-overreaction to environmental antigens. The currently available desensitization schemes and treatments aimed at tipping the immune balance to a more Th1-prone immune response to the respective allergen are not fully effective. Therefore, new approaches to induce a more Th1-oriented immunity to the respective allergen(s) are highly desirable. This could be achieved through targeting the respective allergen to the myeloid dendritic cell of the invention that will be capable of eliciting an effective Th1 response to the allergen (“therapeutic desensitization”). A desensitization vaccine could also be applied to individuals who have a predisposition to develop allergic reaction, but have not yet developed allergic symptoms (“preventive desensitization”).

In another preferred embodiment of the invention the medicament of the invention is for inducing tolerance against an allergen, especially a food allergen. The tolerogenic vaccination may be used for the treatment and/or prevention of allergy. The allergic individual or the individual prone to allergic reactions can be made tolerant to an allergen by targeting the allergen to the myeloid dendritic cells of the invention. The targeted immunogen is an allergen or part of an allergen. The goal is to make the immune system of the individual tolerant to a given allergen.

The tolerogenic vaccination may be also used for the treatment and/or prevention of autoimmune conditions. Many human autoimmune diseases are driven by a Th1 autoimmune process. It would be desirable to make autoimmune individuals or individuals prone to autoimmune reactions tolerant to the autoimmune antigens. The individual would be made tolerant to the autoantigen by targeting the autoantigen to the myeloid dendritic cells of the invention.

For each of the methods of treatment described herein (e.g. vaccines, inducing tolerance, desensitization, anti-tumor preparation, etc.) those of skill in the art will recognize that in some cases, symptoms of the disease or condition that is being treated may be entirely eliminated. For example, vaccine preparations may prevent the establishment of infection by a pathogen, or may prevent the appearance or development of symptoms usually associated with such an infection. However, such treatments may be therapeutically useful even if complete protection does not occur. For example, for example, for some pathogens (e.g. hookworm and other eukaryotic parasites) great advantage may be obtained even by a lessening of symptoms or by the attenuation of symptoms. The same is true for the other applications described herein. For example, it may be highly advantageous to slow the growth of a tumor, even if tumor growth is not entirely prevented, or to reduce or alleviate symptoms of allergic reactions, even if the symptoms do not disappear entirely. Likewise, for vaccines, the invention encompasses compositions which elicit an immune response in a subject, even if the immune response is not protective, but increases or enhances the recipient's ability to combat infection.

The invention will be further illustrated by the following examples. However, these examples should not be interpreted in any way as limiting the scope of the present invention.

### EXAMPLES

### Example 1

### Pathogenic Bacteria and Dead Cells are Internalized by a Unique Subset of Peyer's Patch Dendritic Cells that Express Lysozyme

Material & Methods

Animals and Human Tissue Samples.

6-8 wk-old female/male C57BL/6 or BALB/c mice were from Charles River Laboratories. 8 wk-old male LOU/c rats were from Harlan. lys-EGFP and CX₃CR₁^{GFP} mice were provided by T. Graf (Faust N, Varas F, Kelly L M, et al. Insertion of enhanced green fluorescent protein into the lysozyme gene creates mice with green fluorescent granulocytes and macrophages. Blood 2000; 96:719-26.) and D. Littman (Jung S, Aliberti J, Graemmel P, et al. Analysis of fractalkine receptor CX(3)CR1 function by targeted deletion and green fluorescent protein reporter gene insertion, Mol Cell Biol 2000; 20:4106-14.), respectively. Redundant, surgically resected human Peyer's patch specimens were obtained from patients undergoing surgery for colon cancer. Tissues were obtained with informed patient consent.

Antibodies and reagents. Polyclonal rabbit anti-lysozyme, anti-GFP and anti-Salmonella were from Dako, Torrey Pines Biolabs, and DIFCO respectively. Anti-lysozyme antibody was coupled to Alexa Fluor 647 according to manufacturer's instructions (Invitrogen). Monoclonal antibodies anti-CD11c (N418) and anti-CD11b (M1/70) were from Biolegend. Anti-F4/80 was a hybridoma supernatant. TO-PRO-3, SYTOX blue and Alexa Fluor secondary antibodies were from Invitrogen. Cy3 and Cy5 Goat anti-hamster and control antibodies were from Jackson Immunoresearch. Rhodamine-coupled UEAI was from Vector laboratories. CD8(-Pacific Blue (53-6.7), CD11c-PECy7 (HL3), CD40-FITC (3/23), CD80-FITC (16-10A1) were from BD Bioscience. MHC-II-Alexa700 (M5/114.15.2), CD11b-Pacific Blue (M1/70), CD86-PECy5.5 (GL1) were from eBioscience. F4/80-PE (Clone C1:A3-1) was from AB-Serotec.

Immunofluorescence Staining and Confocal Microscopy.

Peyer's patches or villi from the different part of the small intestine were fixed with 3.2% paraformaldehyde for 1 h, washed in phosphate-buffered saline (PBS), infused overnight in 35% sucrose, and frozen in OCT compound. After permeabilization with 0.5% saponin for 5 min and unspecific binding site blockade with 2% bovine serum albumin (BSA), 1% fetal calf serum (FCS), and 1% donkey or goat serum for 30 min, 8 μm cryostat tissue sections were labeled overnight at 4° C. with primary antibodies or control antibodies followed by incubation for 1 h at room temperature with secondary antibodies and SYTOX Blue or TO-PRO-3 when nuclei staining was required. When 2 rabbit antibodies were used on the same section, AlexaFluor647-coupled anti-lysozyme was incubated at last for 1 hour after the blocking of the donkey anti-rabbit secondary antibodies with 1% rabbit serum for 30 min. The blocking efficiency was checked by the absence of lysozyme staining on villi DC (when used with the anti-EGFP antibody) or on free bacteria (when used with the anti-Salmonella antibody). Slides were mounted in Prolong Gold (Invitrogen) and observed with a Zeiss LSM 510 confocal microscope. Images were analyzed using Adobe Photoshop 7.0 and Imaris 6.1.

DC Isolation.

DCs were isolated from PP, as previously described for lymph nodes DC (Henri S, Vremec D, Kamath A, et al. The dendritic cell populations of mouse lymph nodes. J Immunol 2001; 167:741-8.). Light-density cells were purified by centrifugation on a Nycoprep solution (density of 1.068). CD11c+ cells were sorted using anti-CD11c microbeads and an AutoMACS magnetic cell separator according to manufacturer's instructions (Miltenyi Biotec GmBH).

Flow Cytometry.

Cells were preincubated on ice for 10 min with the 2.4G2 antibody, to block Fc receptors. Intracellular staining was performed with the Cytofix/Cytoperm kit from BD Bioscience following manufacturer's instructions. Multiparameter FACS was performed using a FACSCantoH system (BD Biosciences). FACS data were analyzed using FlowJo software (Tree Star Inc.).

Oral Gavage and Intestinal Loop Infection Model.

Salmonella enterica serovar Typhimurium strain 12023 that did and did not carry the plasmid pGG2, which expresses DsRed from the constitutive promoter rpsM, were incubated in Luria Broth (LB, Difco) supplemented with 50 μg/mL ampiciline overnight at 37° C. with shaking. Bacteria were then diluted 100- or 30-fold in fresh LB and incubated in the same conditions for 2 or 3.5 h and used for oral gavage or intestinal loop infection, respectively. Numbers of bacteria were estimated by measurement of optical density at 600 nm. Bacteria were pelleted and resuspended in PBS at 10⁸ bacteria/mL. After a small incision through the abdominal wall of lys-EGFP mice (starved for 16 h), a loop starting from the ileocaecal junction and containing 2 to 3 Peyer's patches was formed taking care to maintain blood supply. Before closing the loop, 100 μL of PBS containing 10⁷ DsRed-Salmonella was injected. The intestine was then returned to the abdominal cavity for 1 h before the mice were killed and the intestinal loops were removed, opened flat, and washed extensively with PBS before fixation.

Starved C57/BL/6 mice were fed 10⁷ Salmonella in 100 μL PBS. Mice were killed 24 h post-infection and the last 3 PP of the ileum collected and fixed for staining and confocal imaging.

Microsphere Uptake Assay.

CD11c+ magnetic-sorted cells were seeded on Alcian blue-treated coverslips and incubated with 5% FCS supplemented RPMI containing dark red fluorescent 0.39 μm microspheres (1/500; Duke Scientific Corporation) for 1 h at 37° C. before washing, fixation and staining.

Quantitation and Statistical Analysis.

Quantitation was performed on images containing the apex of domes taken from 20 μm cryostat serial sections. Data are given as means −/+ SD. Statistical significance of differences was determined by the unpaired student's t test.

Results:

Lysozyme is Highly Expressed in PP, GC, and SED of Mice, Rats, and Humans:

Lysozyme expression in mouse ileal PP was first investigated by confocal microscopy of tissue sections using a polyclonal antibody that recognizes lysozyme from different species. Strong staining was observed in the SED and in the GC. The IFR was faintly stained whereas the dome-associated villi were mostly devoid of labeling. Few lysozyme-expressing cells penetrated the FAE at the level of M cells, labeled with the lectin UEA-I. As previously described, expression of lysozyme in villi was restricted to the UEA-I-positive Paneth cells of the crypts. In rat and human PP, similar results were obtained except that some lysozyme-expressing cells were also present in the dome-associated villi. With the exception of Paneth cells, conventional villi of rats and humans were not or faintly stained, respectively.

In mice, the lysozyme expressed by Paneth cells, lysozyme P, is distinct from that expressed by the myelomonocytic lineage, lysozyme M, due to gene duplication. To decipher whether cells from mice PP expressed lysozyme M or P isoform, a transgenic mouse was used in which the EGFP gene was inserted into the lysozyme M locus. In the lys-EGFP mice, EGFP expression in place of lysozyme allows the specific detection of myelomonocytic cells. In these mice, a pattern of EGFP fluorescence similar to the staining obtained with the anti-lysozyme antibody was observed, except that Paneth cells were devoid of EGFP fluorescence. Thus, throughout the small intestine, lysozyme M is mainly expressed in the SED and GC of PP whereas lysozyme P is confined to Paneth cells.

Considering that villi contain macrophages and myeloid DC, the absence of detectable lysozyme staining and EGFP fluorescence in villi was surprising. We checked if minimal amounts of lysozyme M were expressed in the villi using an antibody that recognizes EGFP, to increase the detection level of EGFP molecules. This revealed an important cell population that expresses low amounts of EGFP. These cells are hereafter referred to as LysoM^{Lo}, in contrast to the GC, SED, and FAE populations that express high levels of lysozyme, hereafter called LysoM^{Hi}, Although the IFR of PP was only faintly stained for lysozyme, EGFP labeling also revealed an abundant LysoM^{Lo} cell population. To determine whether the FAE, SED, and GC contained LysoM^{Lo} and LysoM^{Hi} cells, lysozyme and EGFP labeling was performed on samples from heterozygous mice (lys-EGFP/+) that express lysozyme M and EGFP. Lysozyme was concentrated in vesicles whereas EGFP was distributed evenly through labeled cells, allowing visualization of dendrites. LysoM^{Lo} cells (no detectable lysozyme staining but EGFP positive) were present in the FAE and SED but not in the GC.

The Main Villous Myeloid Population Expresses CD11b, CX₃CR₁, F4/80, Variable Amounts of CD11c, and Low Levels of Lysozyme:

Although the different myeloid populations of villi have been characterized by flow cytometry, their distribution is not clear because most microscopy studies used single-marker labeling. We took advantage of EGFP detection with an antibody to study their immunophenotype and their distribution in lys-EGFP mice. Villi sections were labelled for the classic myeloid marker CD11b, the DC marker CD11c, and EGFP. All EGFP⁺ cells expressed CD11c except in villous crypt; most also expressed low levels of CD11b)(LysoM^{Lo}CD11c⁺CD11b^{Lo}, whereas high levels of CD11b were only present on round-shape cells that were negative for LysoM and CD11c (LysoM⁻CD11c⁻CD11b^{Hi}). In addition, a minor EGFP⁺ population was negative for CD11b (LysoM^{Lo}CD11c⁺CD11b⁻) and a minor CD11c⁺ population was negative for LysoM and CD11b (LysoM⁻CD11c⁺CD11b⁻). LysoM^{Lo} CD11c⁺CD11b^{Lo} cells were the main myeloid population. They displayed an elongated shape along the villous base to tip axis with dendrites that allowed contact with each other and epithelial cells, thus underlying the architecture of the entire villous. Staining for EGFP and CD11b was regular throughout the villous LP whereas CD11c labeling decreased from the top to the base of the villous, finally disappearing in the crypt vicinity, indicating a regulation of CD11c expression.

F4/80 and CX₃CR₁, 2 widely used markers of macrophages and DC of the villi, were expressed by the major LysoM^{Lo}CD11c⁺CD11b^{Lo} population. F4/80, but not CX₃CR₁, was expressed by the LysoM⁻CD11c⁻CD11b^{Hi} population.

Thus, villi contain at least 4 distinct subpopulations of myeloid and DC cells; the CD11c⁺Lyso^{Lo}CD11b^{Lo}CX3CR1⁺F4/80⁺ subset is the most prevalent.

Myeloid DC that Express Negligible Levels of CD11b are Present in the IFR:

We used the same approach to characterize the LysoM^{Lo} cells of the PP IFR. We observed 5 different subpopulations; the 2 most prevalent were LysoM⁻CD11c⁺CD11b⁻CX₃CR₁⁻F4/80⁻ and LysoM^{Lo}CD11c⁺CD11b⁻CX₃CR₁⁺F4/80⁻. These are likely to correspond to previously described lymphoid DC (CD11c⁺CD11b⁻CD8(⁺) and double-negative DC (CD11c⁺CD11b⁻CD8(⁻) respectively. Expression of LysoM and CX₃CR₁ by some of the latter indicated the myeloid origin of this subset.

LysoM^{Hi} Cells of the GC are Tingible-Body Macrophages:

GC LysoM^{Hi} cells were also analyzed. Since LysoM^{Lo} cells were absent in GC, EGFP staining of lys-EGFP mice was used to identify the whole cell morphology. GC are specialized structures wherein B lymphocytes undergo clonal expansion, class switch recombination, antibody gene diversification, and affinity maturation. Because these B cells proliferate, cell-cycle associated antigens such as Ki67 are good markers of GC. An anti-Ki67 antibody confirmed the localization of the LysoM^{Hi} cells in and around GC. We did not observe any CD11b staining and only faint or no CD11c labeling on these LysoM^{Hi} cells. In lymph nodes, a subset of phagocytes, termed tingible body macrophages, resides in GC and contains many phagocytized apoptotic B cells. We observed that each of the large LysoM^{Hi} cells contained several nuclei, with different stages of apoptosis ranging from condensed nuclei to apoptotic bodies, indicating that LysoM^{Hi} cells in the GC were tingible body macrophages.

LysoM^{Hi} Cells of the FAE and SED are Myeloid DC that Express CD11c, CD11b, CX₃CR₁ but no F4/80:

To distinguish between LysoM^{Lo} and LysoM^{Hi} cells of the SED and FAE, we used EGFP and lysozyme staining in heterozygous lys-EGFP/+ mice. All LysoM^{Lo} (EGFP⁺ but lysoM⁻) and LysoM^{Hi} cells (EGFP⁺ and lysoM⁺) were CD11c⁺. In addition, another CD11c⁺ population that was negative for EGFP and lysozyme was present. In the SED, LysoM^{Hi} cells represented more than 70% of the CD11c⁺ cells.

Also observed was that most SED DC were CD11c⁺CD11b⁺ with a level of CD11b staining similar to that of the CD11b^{Lo} population in the villi. Most FAE CD11c⁺ cells were CD11b⁻. In the SED and FAE, all LysoM^{Hi} cells were CX3CR1⁺ but F4/80⁻. In conclusion, the major DC population of the SED (CD11c⁺CD11b^{Lo}CX₃CR₁⁺F4/80⁻) is the only small intestine DC subset that expresses a high level of lysozyme. These DC are therefore hereafter called LysoDC.

LysoDC Display the Highest Surface Expression of MHC-II and Co-Stimulatory Molecules Among PP DC:

To characterize LysoM^{Hi} cells in more detail, we prepared a DC-enriched fraction from single-cell suspensions of wild-type mice PP²⁰. We performed intracellular immunostaining of lysozyme associated with surface staining of lineage markers. Flow cytometry analyses indicated that LysoM^{Hi} cells represented about 1% of the DC-enriched fraction. At least 80% of LysoM^{Hi} cells expressed CD11c. As expected, the major part of these LysoM^{Hi}CD11c⁺ cells expressed CD11b but no or little Gr1, F4/80, or CD8. Less than 20% of LysoM ^{Hi} cells were negative for all markers tested (CD11b, CD11c, CD8, and (F4/80, Gr1), which indicate the tingible-body macrophage population.

PP DC were identified by their high level of CD11c and intermediate to high level of MHC-II surface expression. 67.6%+/−7.4% of LysoM^{Hi} cells belonged to PP DC. CD11b and lysoM expression defined 3 DC subsets: 48.3%+/−8.2% of PP DC were negative for both CD11b and LysoM, 24.8%+/−8.6% of PP DC expressed CD11b but no LysoM and 16.9%+/−6.3% of PP DC expressed LysoM and a low to high level of CD11b, corresponding to LysoDC. The first subset (negative for CD11b and LysoM) could be subdivided, based on CD8 expression. Importantly, among these DC subsets, LysoDC expressed the highest surface levels of MHC-II, CD40, and CD80 whereas similar levels of CD86 were found in all subsets.

Thus, lysozyme is the first marker of a unique PP DC subset that is mostly present in the SED and expresses high levels of CD11c, MHC-II, CD40, CD80, and CD86.

LysoDC have the Highest Phagocytic Activity Among PP DC:

LysoDC exist in the ideal location to sample luminal antigens transported by M cells. To determine whether they are capable of antigen uptake, we performed a phagocytosis assay, incubating PP CD11c⁺ cells with fluorescent microspheres. Using confocal microscopy, we observed 2 different cell populations that ingested large numbers of microspheres. One was LysoDC, which displayed a high level of CD11c and many dendritic extensions. The other was formed by large oval-shaped cells without dendrites that expressed weak levels of CD11c and no lysozyme. Importantly, among cells that expressed high level of CD11c, only LysoDC contained many microspheres. CD11c⁺ cells incubated with microspheres were also analyzed by flow cytometry. Microsphere fluorescence was correlated to microsphere load. Most LysoDC contained many microspheres. By plotting side scatter (SSC) vs lysozyme for CD11c⁺ cells, 3 subsets could be distinguished: LysoM^{-/Lo}SSC^{Lo} (G1), LysoM^{-/Lo}SSC^{Hi} (G2), and LysoDC (G3). Whereas most of the G1 subset contained no or few microspheres, G2 and G3 subsets internalized many of them Moreover, as observed by microscopy, G2 and G3 subsets differed in their CD11c expression with the SSC^{Hi} subset (G2), showing lower level of surface CD11c, suggesting they were macrophages. Thus. LysoDC displayed the highest phagocytic activity among PP CD11c^{Hi} cells.

LysoDC in Salmonella Typhimurium Uptake:

Because LysoDC were the most efficient PP DC at capturing microparticles in vitro, we investigated whether they were involved in pathogen uptake in vivo. PP are major sites of Salmonella invasion, where these bacteria penetrate M cells. It is not clear what happens to the bacteria once they reach the SED, though local DC have been shown to internalize a Salmonella Typhimurium-attenuated mutant. We infected intestinal loops of lys-EGFP mice with a Salmonella Typhimurium strain that express DsRed. Salmonella were taken up by LysoDC, identified by EGFP fluorescence, within 1 hour. Next, we fed Salmonella to wild-type mice by gavage. 24 h later, Salmonella were found in LysoDC. At this time, bacteria were in the FAE (52.8%−/+8.9% of PP-infected cells) and in the SED LysoDC (40.6%−/+10.4% of PP infected cells). Thus, most Salmonella that crossed the epithelial barrier at the level of PP were taken up by LysoDC.

Involvement of LysoDC in Dead Cell Clearance:

In rats and mice, uncharacterized SED DC are involved in dead epithelial cell engulfment. In the SED of lys-EGFP mice, we occasionally observed in EGFP-labeled cells; instead of a uniform of distribution of EGFP throughout the cytoplasm, we observed large, vacuolar structures devoid of EGFP that contained nuclear debris and apoptotic bodies, indicating that these cells engulfed dead cells. We confirmed that these cells were LysoDC by analyzing wild-type mice in which we did not observe any nuclear debris in other CD11c⁺ subsets. Importantly, the number of LysoDC that contained nuclear debris increased significantly in the upper part of the dome, an area in which epithelial cells die. In this region, M cells disappeared earlier than enterocytes by an unknown mechanism. We investigated whether LysoDC were involved in their clearance by studying this restricted region and observed different steps of engulfment and transport of UEA-I⁺ remnants by LysoDC in the FAE and SED. In the area of M cell disappearance, LysoDC that contained UEAI⁺ remnants represented up to 50% of the LysoDC that contained nuclear debris. In rat tissues, we also observed apoptotic bodies specifically localized in LysoDC. LysoDC therefore appear to be a major DC subset involved in the clearance of dead cells, including M cells.

### DISCUSSION

In this study, a distribution map of the myelomonocytic lineage throughout the small intestine was produced. In villi, the major myeloid subset was identified as CD11c⁺CD11b^{Lo}CX3CR1⁺F4/80⁺. CD11c expression in this myeloid subset varied along the crypt to the top axis of villi, probably representing different maturation/differentiation stages. Lysozyme was not detected in villi myelomonocytic cells, although EGFP staining in lys-EGFP mice revealed slight expression. However in PP, lysozyme was highly expressed by tingible-body macrophages of GC and by an intestinal DC subset called LysoDC that was specifically localized in the SED and FAE. This is the first time that a marker has distinguished a population of intestinal DC exclusively present in the SED of mice, rats, and humans.

Previous studies identified 3 DC subsets in mice PP: lymphoid DC (CD11c⁺CD11b⁻CD8(⁺), localized in the IFR; myeloid DC (CD11c⁺CD11b⁺CD8(⁻) in the SED; and double-negative DC (CD11c⁺CD11b⁻CD8(⁻) at both sites (Iwasaki A, Kelsall B L. Localization of distinct Peyer's patch dendritic cell subsets and their recruitment by chemokines macrophage inflammatory protein (MIP)-3alpha, MIP-3beta, and secondary lymphoid organ chemokine. J Exp Med 2000; 191:1381-94; Iwasaki A, Kelsall B L. Unique functions of CD11b+, CD8 alpha+, and double-negative Peyer's patch dendritic cells. J Immunol 2001; 166:4884-90). We showed that the double-negative DC comprise 2 subpopulations; although one subpopulation expresses little, if any CD11b, it appears to be myeloid based on its expression of 2 other early markers of the myeloid differentiation pathway: CX₃CR₁ and lysozyme (Fogg D K, Sibon C, Miled C, et al. A clonogenic bone marrow progenitor specific for macrophages and dendritic cells. Science 2006; 311:83-7. Ye M, Iwasaki H, Laiosa C V, et al. Hematopoietic stem cells expressing the myeloid lysozyme gene retain long-term, multilineage repopulation potential. Immunity 2003; 19:689-99.). However, as in the villi, detection of lysozyme expression in this subset required EGFP staining of lys-EGFP mice.

Among the different PP DC subsets, LysoDC displayed the highest surface expression level of molecules required for an efficient antigen presentation. Moreover, they are the most efficient PP DC at capturing microspheres in vitro. In vivo, LysoDC are the main DC subset involved in Salmonella Typhimurium uptake. Interestingly, because LysoDC expressed CX₃CR₁ and are therefore distinct from CCR6⁺DC, which were shown to be involved in the activation of Salmonella-specific T-cell response, it indicates a cooperation between these subsets early after infection. It will be important to determine the relationship between these 2 subsets during infection.

LysoDC are also involved in removal of dead cells, including M cells. The engulfment of M-cell remnants by LysoDC could notably allow the capture of infrequent antigens previously sampled by M-cells. Moreover, it explains at least in part how M cells disappear near the top of the dome.

Together, these results indicate that LysoDC, which are ideally located to acquire samples from the gut lumen, possess all the machinery required to efficiently capture and present incoming antigens. Moreover, the increased expression of lysozyme in the specific microenvironment of the SED might protect this major site of antigen sampling against bacterial invasion. Thus, they could represent a first line of innate immune defence as well as a link to adaptive immunity. Further, modulation of lysozyme production may be a mechanism for enhancing protection against bacterial infections in a subject.

### Example 2

### Peyer's Patch Dendritic Cells Sample Antigens by Extending Dendrites Through M Cell-Specific Transcellular Pores

Material and Methods:

Animals.

6-8 wk-old female/male C57BL/6 or BALB/c mice were from Charles River Laboratories. lys-EGFP mice were kindly provided by T. Graf (Faust, N., Varas, F., Kelly, L. M., Heck, S. & Graf, T. Insertion of enhanced green fluorescent protein into the lysozyme gene creates mice with green fluorescent granulocytes and macrophages. Blood 96, 719-726 (2000).).

Antibodies and Reagents.

Polyclonal rabbit anti-ZO-1 and polyclonal rabbit anti-GFP from Torrey Pines Biolabs. Monoclonal antibodies anti-CD11c (N418), anti-JAM-A (BV12) and anti-EpCAM were from Biolegend, SCBT and eBioscience, respectively. TO-PRO-3, SYTOX blue and Alexa Fluor secondary antibodies were from Invitrogen. Cy5 Goat anti-hamster was from Jackson Immunoresearch. Fluorescein and Rhodamine-coupled were from. Vector laboratories.

Immunofluorescence Staining and Confocal Microscopy.

Peyer's patches were fixed with 3.2% paraformaldehyde for 1 h, rinsed in PBS and either microdissected to perform whole-mount dome labeling or infused overnight in 35% sucrose and frozen in OCT compound for cryostat sectioning. After permeabilization with 0.5% saponin for 5 minutes and unspecific binding site blockade with 2% BSA, 1% FCS and 1% donkey or goat serum for 30 minutes, 10 to 20 μm thick cryostat tissue sections were labeled overnight at 4° C. with primary antibodies followed by incubation for 1 h at room temperature with secondary antibodies and SYTOX Blue and/or TO-PRO-3 when nuclei staining was required. Slides were mounted in Prolong Gold (Invitrogen) and observed with a Zeiss LSM 510 confocal microscope. Images of 1024×1024 pixels were then assembled and analyzed using Adobe Photoshop 7.0 and Imaris 6.1.

Results:

LysoDC possess all the machinery required to efficiently capture and present incoming antigens (Lelouard, H. et al. Pathogenic bacteria and dead cells are internalized by a unique subset of Peyer's patch dendritic cells that express lysozyme. Gastroenterology 138, 173-184 e171-173, doi:S0016-5085(09)01746-6 [pii]). LysoDC are the most efficient at capturing microparticles in vitro and display the highest surface expression of class II major histocompatibility complex and costimulatory molecules. Moreover, they are the main DC subset involved in the uptake of the entero-pathogenic bacteria Salmonella Typhimurium. Mostly present in the SED, they are ideally located to acquire samples from the gut lumen. In order to determine how they capture gut luminal antigen we performed two-photon microscopy on explanted PP from a transgenic mouse, the lys-EGFP mouse, in which the EGFP gene was inserted into the lysozyme M locus. This allowed the specific detection of lysozyme M-expressing DC (LysoDC) using EGFP fluorescence. In PP domes viewed from their top, we observed a dense network of fluorescent cells just below the FAE, confirming our previous high-resolution static confocal imaging on cryostat sections. Importantly, based on their dynamic and fluorescence characteristics two populations of cells could be characterized. While the great majority of weakly fluorescent cells in the SED was immobile we found a reduced number of highly mobile and highly fluorescent cells (less than 10% of fluorescent cells) with a mean velocity of 7.0±1.8 μm/min. Their migration paths were weakly restrained (confinement ratio of 0.41±0.18). However, we observed that some of these cells suddenly stopped moving while actively emitting membrane extensions. To determine the position of these discrete cell extensions we imaged the side of the dome to obtain a transversal view of the epithelium. We observed that theses extensions were actively moving into the gut lumen adopting many different shapes. Luminal extensions were connected to the cell body localized in the SED by a cellular thread. The latter was delimited by two membrane constriction points located at the apical and at the basal pole of the epithelium. In some instance, after emitting their extension LysoDC crossed the epithelium. To further document this phenomenon we examined LysoDC luminal extensions by confocal microscopy on cryostat sections of fixed PP of lys-EGFP mice. In addition to the luminal extensions, we observed that some LysoDC were completely exposed to the lumen as demonstrated by the presence of their nuclei at the epithelial surface.

Since M cells are known as the main antigen transcytotic cell of the FAE, we next investigated by confocal microscopy on whole-mount specimens of lys-EGFP mice PP the relationship between M cell and lysoDC. We used the UEA-I lectin and an anti-EGFP antibody to label M cell and LysoDC, respectively. Both transepithelial dendrites and mucosal surface-exposed DC were tightly associated with M cells. In C57Bl/6 mice, villous DC have been shown to express tight junction proteins that allow them to extend paracellular processes across the epithelium while preserving the integrity of the epithelial barrier. Most of these villous transepithelial dendrites display a globular shape and were thus termed balloon-bodies. They are absent in BALB/c mice indicating a strain-restricted phenomenon. By contrast, we observed M cell-specific transepithelial DC extensions in BALB/c mice using CD11c as a DC marker. This indicates that villous- and PP-associated dendrites are distinct. This is further confirmed by the fact that PP DC luminal extensions displayed many different transient shapes clearly distinct from villous balloon-bodies. In addition, PP DC luminal extensions did not reach the intestinal lumen at the lateral borders of M-cells as expected if they crossed the epithelial cells at the level of tight junctions. Instead, the M cell staining surrounded the DC labeling, forming a channel in which the DC extension was inserted. Moreover, DC luminal extension occurred at distance and without disturbance of the tight junctions between M cells and enterocytes as illustrated by a ZO-1 staining, further confirming that PP DC trans-M cell extensions were transcellular rather than paracellular. We also observed the interaction between M cells and LysoDC in vivo by two-photon microscopy of explanted lys-EGFP PP. EGFP fluorescent luminal extensions were observed above M cells. Anchored to the latter by a thin thread, these extensions scanned the surface of and the lumen above M cells.

Transcellular migration has been previously described at the level of the endothelium during leukocyte extravasation by a mechanism known as diapedesis. Transcellular diapedesis involves the recruitment of cell adhesion molecules normally found at the endothelial cell border, namely PECAM-1, CD99 and JAM-A. Although we could not detect PECAM-1 expression in the FAE, we observed that JAM-A was recruited forming a channel that surrounded the DC extension. We also found that the epithelial cell adhesion molecule EpCAM was redistributed at the surface of M cells that present a DC extension and, like JAM-A, formed a channel around the DC extension. Since cell adhesion molecules can mediate homophilic interactions between endothelial cells and migrating leukocytes during diapedesis, we next investigated whether LysoDC expressed JAM-A and EpCAM molecules. JAM-A, but not EpCAM, was strongly expressed at the surface of LysoDC.

In rabbits, M cells express vimentin, an intermediate filament usually absent from epithelial cells. We observed that rabbit leukocytes were capable of emitting transepithelial extensions through M cells and that vimentin was enriched in rings around the luminal extensions, thus indicating that transcellular extensions of DC through M cells may be common to different species and on the other hand that intermediate filaments could be involved in this process.

We next examined on explanted PP of lys-EGFP mice whether these DC extensions were efficient at capturing luminal antigens by adding fluorescent microparticles on the luminal side of the domes. Both top and side views of the dome indicated that LysoDC extensions were actively phagocyting microparticles. Importantly, each LysoDC was able to phagocytose a large number of beads. We also investigated whether LysoDC were able to directly capture entero-pathogenic Salmonella Typhimurium expressing DsRed. We observed that LysoDC extension captured bacteria directly from the lumen and then retracted back to the SED. In the absence of Salmonella we also observed retraction of LysoDC extensions suggesting that this event was independent of the presence of pathogenic bacteria. To determine whether LysoDC could also be shed into the lumen as described for villous DC paracellular migration events, we examined the long-term behaviour of LysoDC extensions. We could monitor ten events of extension retraction but no shedding of LysoDC into the lumen suggesting that LysoDC may play a role in antigen sampling rather than in pathogen exclusion. To determine whether antigens could be taken up by this sampling mechanism before they enter M cells in vivo, we performed a short-term gavage infection with Salmonella Typhimurium which is known to selectively target M cells. Only two hours after gavage Salmonella was found in LysoDC which extend dendrites into the FAE while epithelial cells still were not infected.

Herein a new mechanism of gut antigen sampling is described, which involves a transcellular route through an uncommon epithelial cell type limited in number compared to enterocytes. It may thus allow a fast and accurate sampling of pathogens in a very restricted area, thus limiting the chance of dissemination. Studying the molecular mechanisms involved in this sampling pathway will improve our knowledge on mucosal immune defence and will provide new approaches for oral vaccination targeting.

### REFERENCES

Lelouard H, Henri S, De Bovis B, Mugnier B, Chollat-Namy A, Malissen B, Méresse S, Gorvel J P. Pathogenic bacteria and dead cells are internalized by a unique subset of Peyer's patch dendritic cells that express lysozyme. Gastroenterology. 2010 January; 138(1):173-84.e1-3. Epub 2009 Oct. 1.

Throughout this application, various references describe the state of the art to which this invention pertains. The disclosures of these references are hereby incorporated in entirety by reference into the present disclosure.

1-26. (canceled)

27. A method of delivering a substance of interest to a subject, comprising
administering to said subject an agent which binds to or is taken up by lysozyme-secreting myeloid dendritic cells from the subepithelial dome of Peyer's patches, said substance of interest being associated with said agent.

28. The method of claim 27, wherein said substance of interest is selected from the group consisting of antigens, allergens, tolerogens, adjuvants, drugs, chemicals, DNA, RNA, expression vector systems, engineered viruses, toxins, and enzymes.

29. The method of claim 27, wherein the substance of interest is an anti-tumor agent or an anti-infection agent.

30. The method of claim 27, wherein the substance of interest is a food allergen.

31. The method of claim 27, wherein the subject is a mammal.

32. The method of claim 31, wherein said mammal is selected from the group consisting of a rodent and a human.

33. The method of claim 27, wherein said myeloid dendritic cell is CD11c⁺CD11b^{Lo to Hi}CX3CR1⁺CD8a⁻ with respect to dendritic cell surface marker reactivity.

34. The method of claim 27, wherein said myeloid dendritic cell is CD11c⁺CD11b^{Lo to Hi}CD8a⁻F4/80⁻CX3CR1⁺JAM-A⁺, with respect to dendritic cell surface marker reactivity.

35. The method of claim 27, wherein said myeloid dendritic cell is CD11c⁺BDCA1⁺ with respect to dendritic cell surface marker reactivity.

36. The method of claim 27, wherein said myeloid dendritic cell is genetically engineered to express at least one nucleic acid sequence encoding an expression product.

37. The method of claim 27, wherein said agent is selected from the group consisting of compounds, cells, and microspheres.

38. The method of claim 27, wherein said agent is selected from the group consisting of peptides, petptidomimetics, small organic molecules, antibodies, aptamers and nucleic acids.

39. The method of claim 27, wherein said method is for use as a vaccine.

40. The method of claim 39, wherein the substance of interest is selected from the group consisting of attenuated or dead pathogens, inactivated toxins or immunogenic components thereof, and a protein or a peptide moiety inducing immunity to a pathogen.

41. The method of claim 40, wherein the attenuated or dead pathogens are selected from the group consisting of viruses, bacteria, parasites, fungi and mycoplasma.

42. The method of claim 39, wherein said method further comprises the simultaneous administration of at least one “danger signal”-type adjuvant.

43. The method according to claim 27, wherein said method is for immunogenic or tolerogenic purposes.
